(19) United States
(12) Patent Application Publication
(10) Pub. No.: US 2023/0036911 A1
(43) Pub. Date: Feb. 2, 2023

(54) CULTURE SYSTEMS FOR THE EFFICIENT PRODUCTION OF GENE TRANSFER VECTORS

(71) Applicant: Greffex, Inc., Houston, TX (US)

(72) Inventors: Yan Qi, Lone Tree, CO (US); Uwe D. Staerz, Denver, CO (US)

(21) Appl. No.: 17/758,077

(22) PCT Filed: Dec. 30, 2020
(86) PCT No.: PCT/US2020/067405

§ 371 Date: Jun. 28, 2022

Related U.S. Application Data
(60) Provisional application No. 62/955,002, filed on Dec. 30, 2019.

(51) Int. Cl. C12N 15/86; C12N 5/00

(52) U.S. Cl. CPC C12N 15/86; C12N 5/00; C12N 2710/10343; C12N 2710/10352; C12N 2710/10362

(57) ABSTRACT

The production of gene transfer vectors that have been designed as replication deficient constructs can be inefficient, thus limiting their broad use in medicine. The present invention provides a solution to this problem. It describes how the production efficiencies can be enhanced for gene transfer vectors that are produced by the transfer of DNA and RNA into production cells. The present invention lies m the use of cell cycle control in optimizing the production of gene transfer vectors. The subject of this patent is the modification of cell growth and physiology to enhance the efficiency of vector production. An example is given for the effect of certain media components on the cell cycle and production rate of a fully deleted helper virus independent adenoviral vector. Other applications of this technology are listed.

### CROSS-REFERENCE

This application is an International Application which claims the benefit of priority from U.S. Provisional Application Serial No. 62/955,002 filed on Dec. 30, 2019, the disclosure of which is incorporated herein by reference.

### FIELD

The present invention relates to tissue culture systems used for the encapsidation and/or production of gene transfer vectors, and to their uses for the transfer of nucleic acids into cells, tissues and organs, in particular for applications in gene medicine.

### BACKGROUND

The goal of gene therapy is the cure of diseases by the introduction or repair of disease causing gene defects. Its prime candidates are patients whose diseases are caused by an absence or a dysfunctional version of a given gene. As the human genome was sequenced in its entirety, more diseases have become accessible to gene therapy. Indeed gene therapy has progressed to the clinical trial stage, whose success was stymied by the underlying biology of the gene therapy vectors used.

Gene therapy may use different strategies to deliver or correct the genetic abnormality. The necessary genes may be delivered ex vivo to stem cells, cells, tissues and organs whereupon the modified material is introduced into the body. Alternatively the genetic material is conveyed to cells directly in vivo. In another application, nucleic acids are transferred to cells in vitro mostly for the production of proteins or other biological products.

Different techniques have been developed to introduce genetic material into different cells and cell population. These techniques may be different preparations that include DNA or RNA bound to DEAE-dextran, also compositions of DNA (deoxyribonucleic acid) or RNA (ribonucleic acid) with nuclear proteins, with lipids, with polylysine, with polyethylenimines or polypropylenimines. In addition, DNA packaged with liposomes or the likes are used for delivery. Alternatively viruses engineered as vectors of gene transfer and of vaccination have been investigated. They include modified retroviruses (Rous Sarcoma Virus - RSV, Moloney Leukemia Virus - MLV, HIV-derived - lentivirus, and the like), the Herpes Simplex Virus - HSV, adeno-associated viruses - AAV, adenoviruses - Ad, the Yellow Fever Virus - YF virus, the Vaccinia Virus, and others.

Gene therapy vectors, such as those based on a retrovirus, an AAV, a YF virus and a fully deleted Ad, are produced by transferring DNA or RNA constructs into a eukaryotic cells, such as human embryonic kidney cells - HEK and HeLa cells, in which the respective vectors are assembled. The polyanionic nature of DNA and RNA severely limits their entry into production cells and their transition into the nuclei of these cells. The efficiency of vector production depends on the rate of DNA uptake, the fate of the DNA after cellular uptake, and the status of the production cell. Cellular transfection with naked nucleic acids is inefficient, but can be enhanced by the use of transfection mediators, such preparations of nature lipids and certain cationic polymers. Production cells have to be in a state so that they can use the exogenous DNA to produce the respective gene therapy vector. The exogenous DNA cannot be completed destroyed by cellular DNAse activity, and it has to transition into cellular compartment, such as cellular nuclei, to enable vector production.

Gene transfer vector are delivered to subjects in significant numbers. To facilitate their use in human therapy, it is therefore necessary to develop production systems that deliver gene therapy vectors a high efficiency.

### BRIED DESCRIPTION OF FIGURES

FIG. 1 is a diagram that illustrates an exemplary System of encapsidation of a fully deleted helper virus independent Adenovirus.

FIG. 2 depicts the molecular structure of ascorbic acid.

FIGS. 3A and 3B illustrate the effect of ascorbic acid on the presence of DNA in eukaryotic cells. Specifically, FIG. 3A reflects no addition of ascorbic acid, and FIG. 3B reflects the addition of ascorbic acid. As further described in Example 1, HEK293-type cells that were cultured in the absence of ascorbic acid showed low levels of DNA content indicative a few cells in the S and G2 stages of the cell cycle. In contrast, HEK293-type cells cultured in the presence of ascorbic acid showed increased levels of DNA Therefore, a larger percentage of cells had entered the S and G2 stages of the cell cycle.

FIGS. 4A and 4B illustrate the effect of ascorbic acid on the production of a fully deleted helper virus-independent Adenovirus vector. Specifically, FIG. 4A reflects no addition of ascorbic acid, and FIG. 4B reflects the addition of ascorbic acid. As further described in Example 2, few GFP-expressing cells were seen when Adenoviral vector containing supernatant harvested from cells grown in the absence of ascorbic acid was used to infect the HEK293-type cells. A significantly larger number of infected cells was detected after infection with Adenoviral containing supernatant that was produced in the presence of ascorbic acid.

### DETAILED DESCRIPTION OF PREFERRED EMBODIMENTS

The present invention lies in the use of cell cycle control in optimizing the production of gene transfer vectors. This strategy is being exemplified by, but not limited to the use of ascorbic acid and its derivates as understood to be composed of ascorbic acid as in the formula (1) or formula (II). Other reagents that can be used for this purpose are, but not limited to dehydroascorbic acid, hyrdoxturea, aphidicolin, PD 0332991 HCI, Dinaciclib, AT7519, BS-181 HCI, AZD7762, PF 477736, LY2603618, CHIR-124, and MK-8776.

The ascorbic acids and the other components used in this application may be synthesized chemically or purified by natural components. To optimize the efficiency of vector production the compositions described in the application are added to tissue culture media, in which eurkaryotic packaging cell are cultured. The concentrations of the composition used in the culture media may be adapted by a person skilled in the art in accordance with the composition used for this purpose.

Vector production as understood in this application is the full assembly of a gene transfer vector carrying genetic material of interest. Transfection as understood in this application entails the transfer of polynucleotides coding for genes involved in the production of the vector in question, into eukaryotic cells. Tissue culture medium as understood in this application consists of a water base, to which components are added necessary for the propagation of eukaryotic cells. Vector production as understood in this application consist of the full assembly of a gene transfer vector consisting of a protein capsid or an envelope and a chain or chains of polynucleotides carried within.

In an embodiment of the invention, the gene transfer vectors are derived from a modified retrovirus, such as a Rous Sarcoma Virus (RSV), Moloney Leukemia Virus (MLV), Human Immunodeficiency Virus (HIV), a Herpes Simplex Virus (HSV), an adeno-associated virus (AAV), an adenovirus (Ad), a Yellow Fever Virus (YF virus), a Vaccinia Virus (VV), a Simian Vacuolating Virus (SV), and other natural of synthetic virus adapted to the transfer of genes. They can also an insect, spider or other non-vertebrate virus, such as, but not limited to a baculovirus, or a plant virus, such as, but not limited to a tobacco mosaic virus. They can be used for the in vitro or in vivo transfer of genetic information into eukaryotic cells of human, animal or plant origin. They can be used for the treatment of genetic defects, the enhancement of cellular functions, the induction of differentiation of cells, the death of cells, the induction of cellular functions, the induction of immune responses, and the like.

In an embodiment of the invention, one or more polynucleotides are used to guide the expression of certain genes in cells, into which they have been transfected. In another embodiment of the invention, one or more polynucleotides may encode both the genetic information guiding the assembly of a gene transfer vector and providing the genetic information, such as a certain transgene or certain transgenes, that is or are being used to produce certain molecules, such as, but not limited to proteinaceous products, or RNA molecules with certain functions.

In an embodiment of the invention, these polynucleotides can be either a DNA or RNA of natural of artificial origin, such as double of single stranded DNA or RNA, DNA / RNA hybrid sequences, synthetic or semisynthetic sequences. The nucleic acids can range from oligonucleotides to chromosomes. They can be a single chain or a number of different chains. They may be of human, animal, plant, bacterial, viral, and the like, origin. They may be obtained by any technique known to a person skilled in the art, and in particular by the screening of libraries, by chemical synthesis or alternatively by mixed methods including the chemical or enzymatic modification of sequences obtained by the screening of libraries. They can, moreover, be incorporated into vectors, such as plasmid or other vectors. They may be single- or double-stranded.

In an embodiment of the invention, these polynucleotides may be carried within a gene transfer vector as found in the list above.

In an embodiment of the invention, these polynucleotides may also carry transgenes, such as, but not limited to, therapeutic genes, sequences regulating transcription or replication, antisense sequences, regions for binding to other cell components, and the like. A therapeutic gene is understood, in particular, to mean any gene coding for a proteinaceous product having a therapeutic effect. The proteinaceous product thus encoded can be a protein, a peptide, and the like. This proteinaceous product can be homologous with respect to the target cell (that is to say a product which is normally expressed in the target cell when the latter is not suffering from any pathology). In this case, the expression of a protein makes it possible, for example, to remedy an insufficient expression in the cell or the expression of a protein which is inactive or feebly active on account of a modification, or alternatively to overexpress the said protein. The therapeutic gene may also code for a mutant of a cell protein, having enhanced stability, modified activity, and the like. The proteinaceous product may also be heterologous with respect to the target cell. In this case, an expressed protein may, for example, supplement or supply an activity, which is deficient in the cell, enabling it to combat a pathology, or stimulate an immune response. The therapeutic gene may also code for a protein secreted into the body. Among therapeutic products for the purposes of the present invention, there may be mentioned, more especially, enzymes, blood derivatives, hormones, lymphokines, namely interleukins, interferons, TNF, and the like (FR 92/03120), growth factors, neurotransmitters or their precursors or synthetic enzymes, trophic factors, namely BDNF, CNTF, NGF, IGF, GMF, aFGF, bFGF, NT3, NT5, HARP/pleiotrophin, and the like; apolipoproteins, namely ApoAI, ApoAIV, ApoE, and the like (FR 93/05125), dystrophin or a minidystrophin (FR 91/11947), the CFTR protein associated with cystic fibrosis, tumor-suppressing genes, namely p53, Rb, Rap1A, DCC, k-rev, and the like (FR 93/04745), genes coding for factors involved in coagulation, namely factors VII, VIII, IX, genes participating in DNA repair, suicide genes (thymidine kinase, cytosine deaminase), and the like.

In an embodiment of the invention, the transgenes can also be an antisense gene, genes, sequence or sequences, whose expression in the target cell enables or suppresses the expression of genes or the transcription of cellular mRNAs to be controlled. Such sequences can, for example, be transcribed in the target cell into RNAs complementary to cellular mRNAs and can thus block their translation into proteins.

In an embodiment of the invention, the transgenes can be a protein or a RNA molecules involved in the regulation of the expression and function of other proteins or RNA molecules naturally found in the cell or delivered to the cell by genetic means. The transgenes may code for molecules, such as, but not limited to proteins with antibody or antibody-like binding properties, proteins with enzymatic functions for proteins or other molecules, RNA molecules with binding capacities to proteins and other molecules, and RNA molecules with enzymatic functions for proteins or other molecules.

In an embodiment of the invention, these polynucleotides may also contain one or more genes coding for an antigenic peptide capable of generating an immune response in man or animals. In this particular embodiment, the invention hence makes possible the production either of vaccines or of immunotherapeutic treatments applied to man or animals, in particular against microorganisms, viruses or cancers. Such peptides include, in particular, antigenic proteins specific to the Epstein Barr virus, the HIV virus, the hepatitis B, the influenza virus, the Ebola virus, the Dengue virus, and other medically significant viruses, as well as proteinaceous and other antigens derived from infectious bacteria, such as Bacillus anthracis, Mycobaterium tuberculosis and other medically significant bacteria, as well as other infectious diseases, such as but not limited to malaria, as well as proteinaceous antigens associated with tumors, against beneficial immune responses can be raised.

In an embodiment of the invention, these polynucleotides may compromise sequences permitting the expression of the therapeutic or antigenic genes. These sequences can be the ones, which are naturally responsible for the expression of the genes in question or can be of different origin. In particular they can be promotor sequences of eukaryotic or viral genes. In this connection, the promoters of the EIA, MLP, CMV, RSV and the like, genes may, for example, be mentioned. In addition, these expression sequences may be modified by the addition of activation or regulatory sequences or sequences permitting a tissue-specific expression or an enhanced expression.

In an embodiment of the invention, these polynucleotides may carry a nucleic acid sequence that can also contain, especially upstream of the therapeutic gene, a signal sequence directing the therapeutic product synthesized into the pathways of secretion of the target cell. This signal sequence can be the natural signal sequence of the therapeutic product, but it can also be any other functional signal sequence, or an artificial signal sequence. Moreover, in addition, the nucleic acid can also contain, especially upstream of the therapeutic gene, a sequence directing the therapeutic product synthesized towards a preferential cellular compartment, such as a nuclear localization sequence.

In an embodiment of the invention, the eukaryotic cells used for the production of a gene transfer vector are a line of human cells, such as, but not limited to HEK 293 cells, HeLa cells, A549, and the likes, or primary human cells harvested from different tissues. These cells can be derived from animal origin, such as, but not limited to Chinese hamster ovary cells, Vero cells, and the likes. They can be derived from insects, spiders or non-vertebrates, such as, but not limited to sf9 cells, and the likes. They can be derived from plants.

In an embodiment of the invention, transfections of nucleic acids are achieved by the addition of a single or number of polynucleotides to cultures of eukaryotic cells with the goal that the polynucleotides enter these cells. Transfection rates may be enhanced by the addition of certain compounds to the polynucleotides upon addition of the cultures of eukaryotic cells. These compositions comprise, in addition, an adjuvant capable of combining with the polymer/nucleic acid complex and of improving the transfecting power, such as certain adjuvants (lipids, proteins, lipopolyamines, synthetic polymers, for example) capable of combining with the polymer/nucleic acid complex. These adjuvants may be, but are not limited to, CaCl2, Lipofectamine, Roche X-tremeGENE, JetPEI, and the likes.

In an embodiment of the invention, these polynucleotides are provided as one of more chains of DNA molecules linear or circular in composition. They may be single of double stranded DNA molecules. They may be one or more chains of RNA molecules linear or circular in composition. They may be single of double stranded RNA molecules. They may be delivered as combinations of DNA and / or RNA molecules of different composition, single or double stranded, linear or circular molecules. They may be hybrid molecules composed of DNA and RNA molecules of different composition, single or double stranded, linear or circular molecules. They may be harvested from natural sources, such as, but not limited to, bacteria, animal cells, animal tissue, viruses, plant cells, plant tissues, and the likes. They may be synthesized by techniques known in the art.

In an embodiment of the invention, the growth behavior of the eukaryotic cells used for the production of a gene transfer vector is controlled by seeding different cell numbers, by the length of culture time, by the culture medium, by the culture temperature, by the culture vessel, and by other possible variation in the cell culture procedure.

In an embodiment of the invention, the cell behavior of the eukaryotic cells used for the production of a gene transfer vector is modified by the addition of different compounds, such as, but not limited to ascorbic acid, dehydroascorbic acid, hyrdoxturea, aphidicolin, PD 0332991 HCI, Dinaciclib, AT7519, BS-181 HCI, AZD7762, PF 477736, LY2603618, CHIR-124, MK-8776 and the likes. The cell behavior may be modified by the time of addition of such compounds during the culturing of the eukaryotic cells used for the production of a gene transfer vector. Addition of such compounds may occur prior, during or after the transfection of the eukaryotic cells used for production of gene transfer vector with nucleic acids that carry gene guiding the production of gene transfer vectors. Eukaryotic cells used for the production of a gene transfer vector may be exposed to such compounds for a limited period of time followed by the removal of such compounds, or for extended periods of time during their culture. They may be exposed to a single such compound for more than one culture period. They may be exposed to more than one such compound added to culture at the same time or at different times.

In an embodiment of the invention, the cell behavior of the eukaryotic cells used for the production of a certain molecules, such as proteins or RNAs, is modified by the addition of different compounds, such as, but not limited to ascorbic acid, dehydroascorbic acid, hyrdoxturea, aphidicolin, PD 0332991 HCI, Dinaciclib, AT7519, BS-181 HCI, AZD7762, PF 477736, LY2603618, CHIR-124, MK-8776 and the likes. The cell behavior may be modified by the time of addition of such compounds during the culturing of the eukaryotic cells used for the production of a gene transfer vector. Addition of such compounds may occur prior, during or after the transfection of the eukaryotic cells used for production of gene transfer vector with nucleic acids that carry gene guiding the production of gene transfer vectors. These eukaryotic cells may be exposed to such compounds for a limited period of time followed by the removal of such compounds, or for extended periods of time during their culture. They may be exposed to a single such compound for more than one culture period. They may be exposed to more than one such compound added to culture at the same time or at different times.

In an embodiment of the invention, the produced gene transfer vectors are released into the culture medium by the eukaryotic cells used for the production of a gene transfer vector. They may be found within the eukaryotic cells used for the production of a gene transfer vector and may be release from these cells by different forms of cell lysis, such as, but not limited to, low incubation temperature, addition of a detergent. They may be found in the culture medium of and within the eukaryotic cells used for the production of a gene transfer vector. They may have to undergo an enrichment and/or a purification process before they are used for their intended applications.

### EXAMPLES

The present invention will be described more completely by means of the examples, which follow, which are to be considered as illustrative and non-limiting.

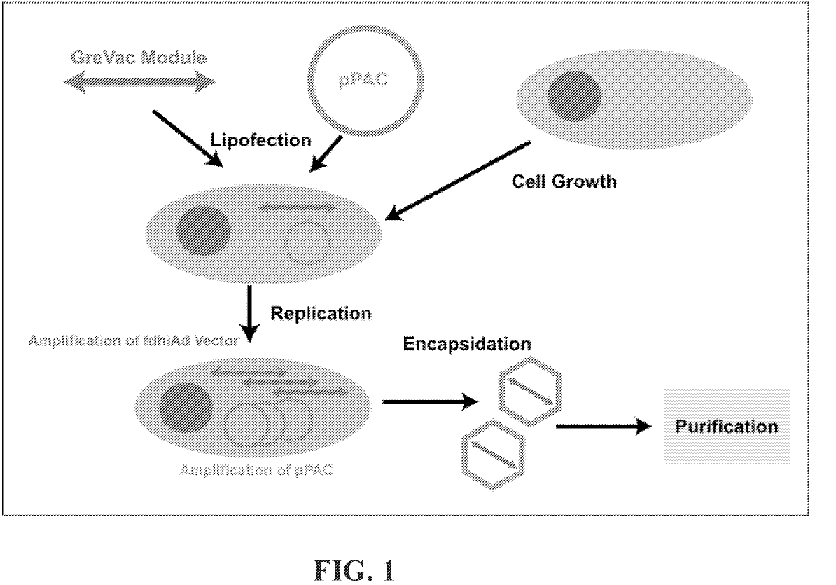

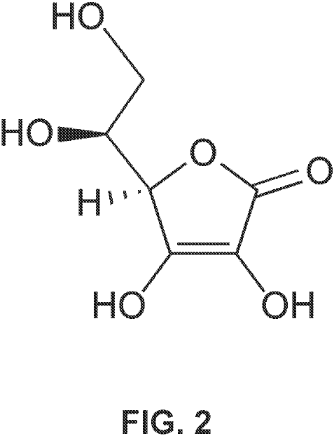

In the present example, the use of ascorbic acid composition exemplified by its used for the production of DNA virus, such as, but not limited to, a fully deleted helper virus independent Adenoviral vector. The vector production systems is comprised of three components (as exemplified in FIG. 1): (i) The GreVac module is packaged into the vector. It carries an expression cassette for a transgene, such as the green fluorescent protein, (cytomegalovirus immediate early promotor/enhancer / transgene / human growth hormone poly-adenylation sequence) flanked by ITRs and Ψ sequences, and is called fdhiAdGFP. A non-coding internal fragment of the human 5-aminoimidazole-4-carboxamide ribonucleotide formyltransferase gene is used as “stuffer” to fill in for the deleted Ad genome. (ii) The pPAC5 packaging plasmid provides Ad late (L1, L2, L3, L4, L5), and E2 and E4 genes for replication and packaging of GreVac vector modules. (iii) The HEK293-type host cells that are based on HEK293 human cells. They carry the genes for Ad EIA and pIX.

### Example 1 - Increase of Cellular DNA by the Addition of Ascorbic Acid to Cultured Cell Culture

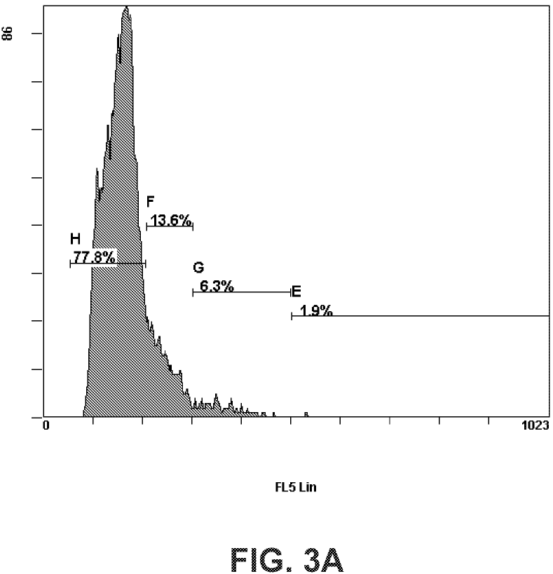

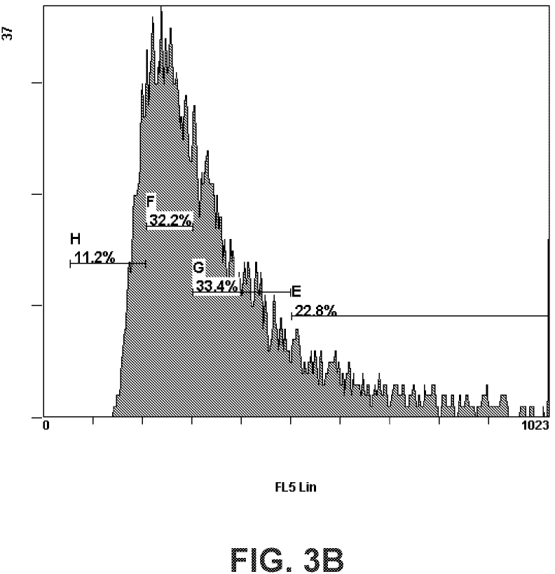

HEK293-type cells were taken from a working cell bank and were cultured in an appropriate tissue culture vessel in an appropriate tissue culture medium both known by a person skilled in the art. They were expanded at appropriate conditions, such 37° C., 5% CO₂. Cell growth and density was monitored daily. One day after cellular confluency was reached the cells were harvested by methods known by a person skilled in the art. The harvested HEK293-type cells were diluted in tissue culture medium and reseeded in an appropriate tissue culture vessel in and appropriate tissue culture medium. To one set of HEK293-type cells ascorbic acid was added at a final concentration of 5 µg/ml. After 24 hrs of culture, the cells were harvested and stained for DNA content by Vybrant (Invitrogen) following the manufacturers protocols. The cells were analyzed by fluorescence on a fluorescence activate cell analyzer (Beckman-Coulter Cytomics FC500). As exemplified in FIGS. 3A and 3B, HEK293-type cells that were cultured in the absence of ascorbic acid showed low levels of DNA content indicative a few cells in the S and G2 stages of the cell cycle. In contrast, HEK293-type cells cultured in the presence of ascorbic acid showed increased levels of DNA. Therefore, a larger percentage of cells had entered the S and G2 stages of the cell cycle.

### Example 2 - Production of a Fully Deleted Helper Virus Independent Adenoviral Vector that Carries the Gene for Green Fluorescent Protein as Transgene

HEK293-type cells were taken from the working cell bank and were cultured in an appropriate tissue culture vessel in an appropriate tissue culture medium both known by someone experienced in the art. They were expanded at 37° C., 5% CO₂. Cell growth and density were monitored daily by light microscopy. One day after cellular confluency is reached the cells were harvested by methods known by someone experienced in the art. The harvested HEK293-type cells were diluted in tissue culture medium and reseeded in an appropriate tissue culture vessel in and appropriate tissue culture medium. To one set of HEK293-type cells ascorbic acid were added at a final concentration of 5 µg/ml. The HEK293-type cells were transfected with the linearized DNA of the fdhiAd GFP vector module and DNA of a pPAC5 packaging plasmid together with a transfection mediator such as JetPEI (PolyPlus) according to the manufacturer’s protocol. The transfected cells were cultured at for an appropriate time, such us 5 days, under appropriate conditions, such as 37° C., 5% CO₂. Then, the cells were harvested. They were centrifuged and resuspended in a modified PBS medium. The encapsidated Adenoviral vectors were released from the cells. The cells were frozen at minus 80° C., then thawed at room temperature. This cycle was repeated. The cellular debris was removed by centrifugation and the supernatant containing released Adenoviral vectors is collected.

To measure the efficiency of encapsidation, HEK293-type cells in culture were infected with defined volumes of the Adenoviral vector containing supernatants. The transduced cells were incubated for 2 days at 37° C., 5% CO₂. Then they were harvested and examined for the extent of green fluorescence on a fluorescence activate cell analyzer (Beckman-Coulter Cytomics FC500) as an indicator of the number of infectious adenoviral particles released from the production cells.

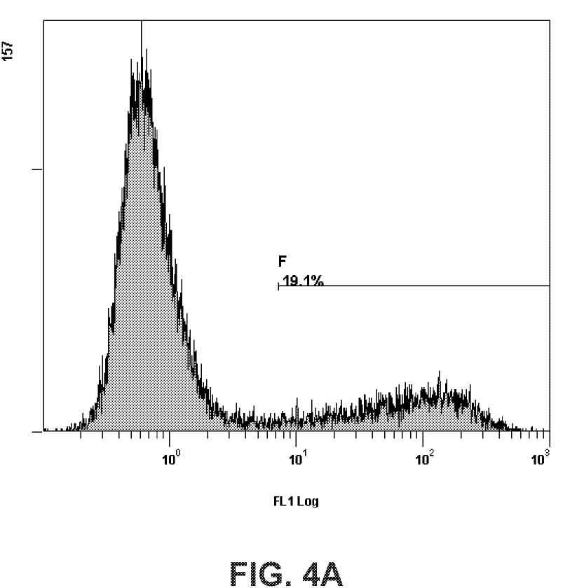

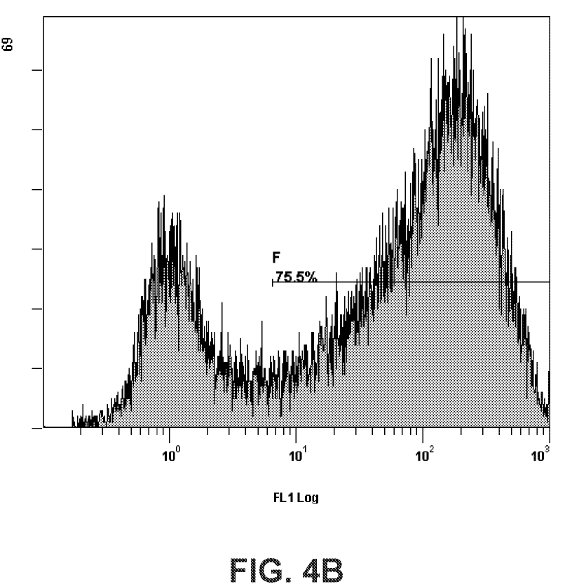

As exemplified in FIGS. 4A and 4B, few GFP-expressing cells were seen when Adenoviral vector containing supernatant harvested from cells grown in the absence of ascorbic acid was used to infect the HEK293-type cells. A significantly larger number of infected cells was detected after infection with Adenoviral containing supernatant that was produced in the presence of ascorbic acid.

These studies indicated that changes in the cell behavior mediated by the addition of ascorbic acid had increased the rate of encapsidation of a gene transfer vector, such a fully deleted helper virus independent Adenoviral vector, produced by transfection of polynucleotides that guided the vector assembly and carried a transgene.

### DEFINITIONS

Unless otherwise explained, all technical and scientific terms used herein have the same meaning as commonly understood by one of ordinary skill in the art to which this disclosure belongs. Definitions of common terms in the molecular biology may be found in Benjamin Lewin, Genes V, published by Oxford University Press, 1994 (ISBN 0-19-854287-9); Kendrew at al. (eds.), The Encyclopedia of Molecular Biology, published by Blackwell Press Ltd, 1994 (ISBN 0-632-02182-0); and Robert A. Meyers (ed.), Molecular Biology and Biotechnology: a Comprehensive Desk Reference, published by VCH Publishers, Inc., 1995 (ISBN 1-56081-569-8). Definition of terms in chemistry may be found in McGraw-Hill, Dictionary of Chemistry, 2003 (ISBN 0-07-141046-5).

As used herein, the term “polymer” refers to a chemical compound or mixture of compounds consisting of repeating structural units created through a process of polymerization.

As used herein, the term “synthesis” refers chemical synthesis in which chemical reactions are purposeful executed to obtain a product or several products.

As used herein, the terms “nucleic acid”, “nucleic acid molecule” and “polynucleotide” include both deoxyribonucleic acid (DNA) and ribonucleic acid (RNA) and, unless otherwise specified, includes both double-stranded and single-stranded nucleic acids. Also included are molecules comprising both DNA and RNA, either DNA/RNA heteroduplexes, also known as DNA/RNA hybrids, or chimeric molecules containing both DNA and RNA in the same strand. Nucleic acid molecules of the invention may contain modified basis. The present invention provides for nucleic acid molecules in both the “sense” orientation (i.e. in the same orientation at coding strand of the gene) and in the “antisense” orientation (i.e. in an orientation complementary to the coding strand of the gene).

As used herein, DNA may be introduced into a cell by processes referred to as “transfection” or “transformation”. Transfection refers to the introduction of genetic material across the membrane of a eukaryotic cell by chemical, mechanical or physical means. Transformation refers to the introduction of genetic material into non-eukaryotic cells, such as bacteria by chemical, mechanical or physical means.

As used herein, the term “cell” refers to cells derived from eukaryotes which are organisms whose cells contain complex structures enclosed within membranes as well as nuclei and other organelles and that are formally referred to as the toxin eukarya.

As used herein, the terms “polypeptide” and “protein” refer to any chain of amino acids regardless of the length or post-translational modification (for example, glycosylation or phosphorylation), such as an unmodified protein or a fragment or segment of a protein.

As used herein, the term “antigen” refers to any compound, composition, or substance than can stimulate the production of antibodies, or a T cell response in an animal or a human. The term “antigen” includes all related antigenic epitopes. “Epitope” refers to a site on an antigen to which antibodies and T cells respond.

As used herein, the term “promoter” is intended to mean a regulator region of DNA usually comprising a TATA box capable of directing DNA polymerase II to initiate RNA synthesis at the appropriate transcription initiation site for a particular coding sequence. A promotor may additionally comprise other recognition sequences, but not limited to, referred to as upstream promotor elements, which influence the transcription initiation rate. The term “constitutive promotor” refers to a promotor that allows for transcription of its associated gene.

As used herein, the term “gene” refers to a DNA sequence that either directly or indirectly encodes a nucleic acid or protein product that has a defined biological activity.

As used herein, the term “transgene” refers to a genetic sequence that is carried on a polynucleotide that is transferred into a cell.

It is appreciated that several other features and functions, or alternatives thereof, may be desirably combined into many other systems or applications. Various presently unforeseen or unanticipated alternatives, modifications, variations, or improvements therein may be subsequently made by those skilled in the art which are also intended to be encompassed in the present invention.

What is claimed is:

1. A method for the enhanced production of gene transfer vectors comprising: (a) providing a vector production cell; (b) transfecting, into the production cell, one of more genetic constructs, coding for the vector genome and the vector production information; and (c) an agent able to induce an arrest of the cell cycle of the vector production cell.

2. The method of claim 1 wherein the gene transfer vector is based on a DNA virus.

3. The method of claim 1 wherein the gene transfer vector is based on an RNA virus.

4. A method of claim 1 wherein the vector production cell is an animal cells.

5. A method of claim 1 wherein the vector production cell is a human cell.

6. A method of claim 1 wherein the vector production cell is an insect cell.

7. A method of claim 1 wherein the vector production cell is a fungal cell.

8. A method of claim 1 wherein the agent to induce an arrest of the cell cycle of the vector production cell is selected from the group consisting of dehydroascorbic acid, hyrdoxturea, aphidicolin, PD 0332991 HCI, Dinaciclib, AT7519, BS-181 HCI, AZD7762, PF 477736, LY2603618, CHIR-124, and MK-8776.

9. A method of claim 1 wherein the addition of an agent added to induce cell cycle arrest of the vector production cell is timed.

10. A method for the enhanced production of an adenoviral gene transfer vector comprising: (a) providing a human vector production cell; (b) transfecting, into the production cell, modified adenoviral genome; and (c) dehydroascorbic acid as agent able to induce an arrest of the cell cycle of the vector production cell.

11. A method of claim 10 wherein the vector production cell is a HEK293 derived cell.

12. A method of claim 10 wherein the vector production cell is a cell that carries genes of an adenoviral E1 region.

13. A method of claim 10 wherein the adenoviral gene transfer vector is a partially deleted adenoviral vector.

14. A method of claim 10 wherein the modified adenoviral genome is deleted of all adenoviral genes.

15. A method of claim 10 wherein the modified adenoviral genome is comprised of a construct of an adenoviral genome deleted of all adenoviral genes and second constructs providing the packaging information for adenoviral genome.
